(19) United States
(12) Patent Application Publication
(10) Pub. No.: US 2018/0055448 A1
(43) Pub. Date: Mar. 1, 2018

(54) CONTACT LENS FOR ANALYZING OCULAR FLUID

(71) Applicant: KONINKLIJKE PHILIPS N.V., Eindhoven (NL)

(72) Inventors: KORAY KARAKAYA, NOORD BRABANT (NL); SUSANNE MAAIKE VALSTER, VALKENSWAARD (NL); SIMA ASVADI, EINDHOVEN (NL); RON MARTINUS LAURENTIUS VAN LIESHOUT, GELDROP (NL); KIRAN HAMILTON J. DELLIMORE, EINDHOVEN (NL); CHARLES FREDERIK SIO, EINDHOVEN (NL); MAARTEN PETRUS JOSEPH KUENEN, VELDHOVEN (NL)

(21) Appl. No.: 15/561,527

(22) PCT Filed: Feb. 19, 2016
(86) PCT No.: PCT/EP2016/053609

§ 371 Date: Sep. 26, 2017

(30) Foreign Application Priority Data
Mar. 26, 2015 (EP) 15160979.9

(51) Int. Cl. A61B 5/00; A61B 5/145; A61B 3/10; A61B 5/01; A61B 10/00

(52) U.S. Cl. CPC A61B 5/6821; A61B 5/14507; A61B 5/14532; A61B 5/14539; A61B 5/14546; A61B 2010/0019; A61B 5/01; A61B 5/0004; A61B 10/0012; A61B 5/4809; A61B 3/101

(57) ABSTRACT

There is provided a contact lens for detecting changes in a property of ocular fluid. The contact lens comprises a lens part comprising an indicator material, wherein the volume of the indicator material is variable in dependence on a property of ocular fluid. The contact lens further comprises output means disposed on the lens part, wherein the output means is configured to provide an output which is variable in dependence on the volume of the indicator material.

### TECHNICAL FIELD OF THE INVENTION

The invention relates to a contact lens for detecting changes in a property of a fluid on an eye of a subject.

### BACKGROUND TO THE INVENTION

Fluids which may be present on an eye of a subject include tears, discharge from the eye, mucus and meibum, among others. Hereinafter the term “ocular fluid” is used to refer to any fluid or mixture of fluids on an eye of a subject. Ocular fluid is generally made up of fluid secreted by the lacrimal glands in the eye, and plasma components which have leaked either across the blood-tear barrier or from tissue interstitial fluid. It has a complex composition, containing soluble and insoluble mucins, proteins, enzymes and aqueous components, covered by an upper lipid layer. Changes to the chemical composition of ocular fluid can be caused by internal factors (e.g. disease, reaction to a drug, etc.). Ocular fluid can therefore be a source for analyses of trace constituents (analytes) in a body fluid, and can thereby play a role in disease diagnosis and/or in monitoring the body's response to therapeutic drugs. Changes to the chemical composition of ocular fluid can also be induced by environmental factors (e.g. air pollution), so the analysis of ocular fluid can also be useful in determining how a subject's environment might be influencing their health.

Typically ocular fluid is analyzed by collecting a sample and then analyzing the sample in a lab to detect a target substance of interest, e.g. using liquid chromatography, enzymatic assays, etc. However; obtaining useful samples of ocular fluid is difficult. Under normal conditions each eye contains 7-10 μl of ocular fluid, but this volume is normally less for aging people, particularly if they suffer from conditions such as “dry eye.” Thus, to collect a sufficient volume of ocular fluid for analysis, it is often necessary to artificially stimulate tear production, e.g. with tear-inducing chemicals, fans, etc. The ocular fluid is typically collected using a capillary tube made from glass or silicone. However; ocular fluid collection by capillary tubes is invasive and irritating and can damage the eye if not carefully done. Furthermore, it has been shown that composition of ocular fluid that results from mechanical or chemical eye stimulation differs from the composition of normally secreted ocular fluid (e.g. because the concentration of some constituents of ocular fluid is flow-dependent). Another shortcoming of existing ocular fluid analysis techniques is that, because of the difficulties involved in the sample collection and the time and effort required for the lab analysis of each sample, they can only be used for obtaining point data (rather than for continuous monitoring) and are unsuitable for providing information about the variability of ocular fluid composition over short time periods (i.e. less than a day) or during the night. This is a particular problem in relation to analytes which are subject to 24 hour variations (such as melatonin, which is of interest in relation to sleep disorders) and/or have a short half time, because in such cases the concentration is a function of the time of sample collection.

Some of these issues are addressed in US 2014/0107445, which describes a system for “in-eye” analysis of ocular fluid based on monitoring the electrical properties of the ocular fluid. The system uses a contact lens, in which is embedded a two-electrode electrochemical sensor, control electronics, and an antenna for wirelessly indicating the amperometric current measured by the sensor. The method enables relatively unobtrusive measurement of some tear film properties. However, the electrical properties of ocular fluid in general are associated with the general particle concentration and osmolarity of the fluid, meaning that it is difficult or impossible to determine the concentrations of specific analytes in the ocular fluid using this system.

There is therefore a need for a system which is able to non-invasively determine the concentration of particular target analytes in ocular fluid. Preferably such a system would permit continuous or near-continuous monitoring of the concentration, would be low-cost and simple to use, and would be suitable for use with elderly people or others who naturally have low volumes of ocular fluid.

### SUMMARY OF THE INVENTION

According to a first aspect of the invention, there is provided a contact lens for detecting changes in a property of ocular fluid. The contact lens comprises a lens part comprising an indicator material, wherein the volume of the indicator material is variable in dependence on a property of ocular fluid. The contact lens further comprises output means disposed on the lens part, wherein the output means is configured to provide an output which is variable in dependence on the volume of the indicator material.

In some embodiments the property comprises one or more of: the presence of a target analyte, the concentration of a target analyte, pH, volume, osmolarity, a ratio of compounds in the ocular fluid; evaporation rate; viscosity; rheology; tear film stability; temperature; density.

In some embodiments in which the property comprises the concentration of a target analyte, the indicator material is arranged to absorb the target analyte and the volume of the indicator material is variable in dependence on the amount of the target analyte contained in the indicator material. In some such embodiments the target analyte comprises one of: glucose; an amino acid; an organic acid; a fatty acid, a polyol; a hormone; a protein, a metabolite, an enzyme, a nucleic acid, a lipid, an electrolyte, a chemical induced by medication intake; an environmental pollutant.

In some embodiments the indicator material comprises one or more of: a bio-responsive material, wherein the volume of the bio-responsive material is variable in dependence on the presence and/or concentration of a target biological agent; and an environmentally-responsive material, wherein the volume of the environmentally-responsive material is variable in dependence on an environmental factor. In some embodiments the indicator material comprises one or more of: a molecularly imprinted polymer; and a hydrogel.

In some embodiments the output means comprises a radio frequency (RF) antenna disposed on the indicator material such that a change in the volume of the indicator material causes a change in the strain experienced by a conductive part of the antenna. In some such embodiments the RF antenna is configured such that a transfer function of the RF antenna is variable in dependence on the strain experienced by the conductive part of the antenna.

There is also provided, according to a second aspect of the invention, a system for detecting changes in a property of ocular fluid. The system comprises a contact lens according to the first aspect. The system further comprises a reader arranged to detect the output from the output means.

In some embodiments the reader is arranged to detect the output without being in contact with the contact lens.

In some embodiments the system further comprises a processing unit arranged to determine a value of the property based on the output detected by the reader. In some such embodiments the reader is arranged to continuously detect the output and the processing unit is arranged to determine a time-series of values of the property. In some embodiments the processing unit is further arranged to generate at least one output signal based on the determined value. In some such embodiments the at least one output signal comprises one or more of: a signal arranged to cause the determined value to be shown on a display of the reader; a signal arranged to cause the determined value to be shown on a display of a remote device; a message to a portable device of a caregiver containing the determined value; a data transmission to a memory of the reader; a data transmission to a remote server. In some embodiments the processing unit is comprised in the reader.

In some embodiments in which the output means of the contact lens comprises an RF antenna disposed on the indicator material, the reader is further arranged to detect the output by: transmitting RF energy to the contact lens; and in response to transmitting RF energy to the contact lens, receiving RF energy from the RF antenna.

In an embodiment, it is provided a contact lens for detecting changes in a property of ocular fluid, the contact lens comprising: a lens part comprising an indicator material, wherein the volume of the indicator material is variable in dependence on a property of ocular fluid; and an output means disposed on the lens part, wherein the output means comprises an RF antenna disposed on the indicator material such that a change in the volume of the indicator material causes a change in the strain experienced by a conductive part of the RF antenna and/or a change in the configuration of the RF antenna, such that a signal transmitted by the RF antenna is variable in dependence on the volume of the indicator material.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a top view of a contact lens, according to a general embodiment;

FIG. 2a is a cross-sectional view of a part of a contact lens in a first state, according to a first specific embodiment;

FIG. 2b is a cross-sectional view of the contact lens part of FIG. 2b in a second state;

FIG. 3a is a top view of the contact lens of FIG. 2a having a first antenna configuration;

FIG. 3b is a top view of the contact lens of FIG. 2a having a second antenna configuration;

FIG. 4 is a schematic of a system for detecting changes in a property of ocular fluid, according to a second specific embodiment;

FIG. 5 is a flow chart illustrating a method for determining the concentration in ocular fluid of a target analyte using the system of the second specific embodiment; and

FIG. 6 is a graph showing the antenna response to a resistance change of a contact lens antenna according to the second specific embodiment.

### DETAILED DESCRIPTION OF THE EMBODIMENTS

Embodiments of the invention seek to enable the unobtrusive, continuous measurement of ocular fluid characteristics, including the concentration in ocular fluid of one or more target analytes. In particular embodiments this is achieved by providing a sensor in the form of contact lens that undergoes a physical response, in the form of a volume change (i.e. swelling/shrinking), to changes in one or more ocular fluid properties (e.g. concentration of a target analyte, ocular fluid amount, pH, etc.). In some embodiments a reader device that is able to read a response of the sensor remotely (i.e. without requiring contact between the reader and the sensor) is also provided. In some embodiments the reader device is arranged to be worn on a body part of the user. In some such embodiments the reader device comprises, or is attachable to, a pair of spectacles. In some embodiments the reader device comprises, or is attachable to, an item of wearable head gear, such as a head band, headphones, a hat, etc. In some embodiments the reader device comprises, or is attachable to, an item of gear arranged to be worn on a body part other than the head, such as a wrist band, watch, arm band, neck brace, necklace, etc.

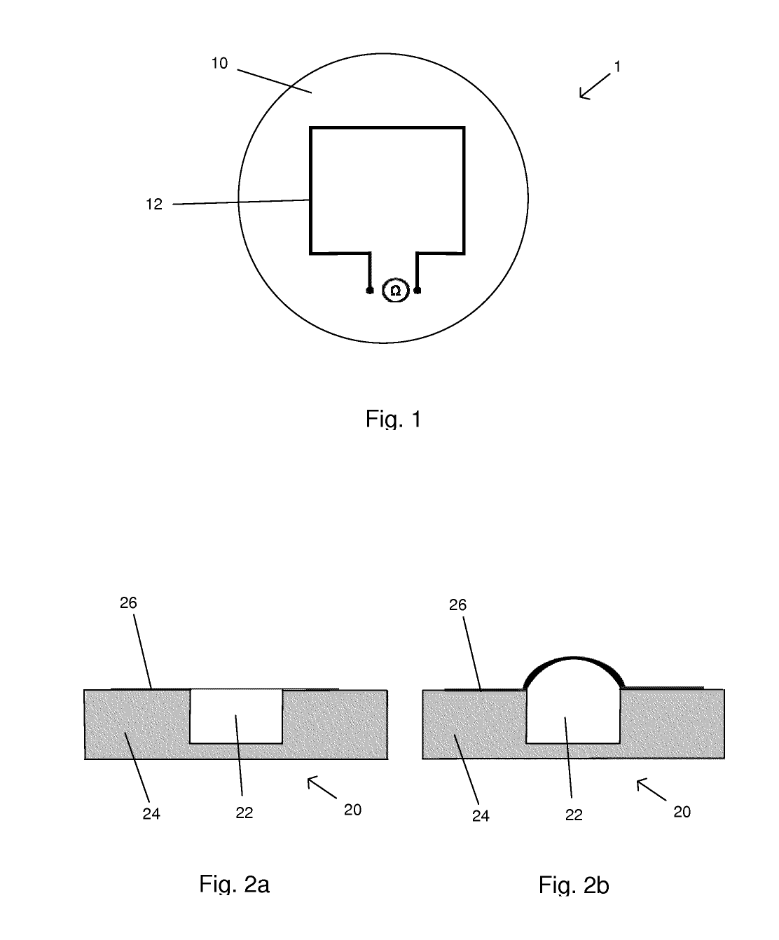

FIG. 1 shows a contact lens 1 according to a general embodiment of the invention. The term “contact lens” should be understood to refer to any device that is suitable for wearing in or on the eye for an extended period of time. It should be appreciated that contact lenses according to embodiments of the invention are preferably, but need not be, transparent. The contact lens 1 comprises a round lens part 10 with a concave curvature configured to mount to a corneal surface of an eye. The lens part 10 comprises an indicator material, the volume of which is variable in dependence on a property of ocular fluid. In some embodiments the entire lens part 10 is formed from the indicator material. In some embodiments the lens part 10 is partially formed from the indicator material and partially formed from another material, e.g. a conventional contact lens material. In some embodiments the lens part 10 is shaped to provide a predetermined, vision-correcting optical power, in the manner of a conventional contact lens. It will be appreciated that the stiffness of the indicator material (and, if present, the non-indicator material comprised in the lens part), and/or the arrangement of the indicator material (and, if present, the non-indicator material) can be selected to achieve a particular shape of the indicator material when it is in a swollen state. For example, in some embodiments it is advantageous to maximize the swelling in the out-of-plane direction (i.e. perpendicular to the surface of the eye).

In some embodiments the indicator material comprises a material which changes volume in response to the amount of ocular fluid present in the users eye. In some embodiments the indicator material comprises a material which changes volume in response to changes in a chemical property of ocular fluid with which the indicator material is in contact. In some such embodiments the property of the ocular fluid comprises one or more of: the concentration of a target chemical; the salt concentration; a ratio of compounds in the ocular fluid; pH; volume; osmolarity; evaporation rate; viscosity and rheology; tear film stability; temperature; and density. In some embodiments the indicator material comprises a material which changes volume in response to changes in a biological property of ocular fluid with which the indicator material is in contact. In some such embodiments the property of the ocular fluid comprises one or more of: the presence of a target biological agent, the concentration of a target biological agent, a property of a target biological agent. A target biological agent can comprise, for example, a protein, a lipid, a hormone, a goblet cell, a mucin, a bacteria, a virus, etc.

Various materials which exhibit a volume change in response to a change in an environmental factor are known in the art. For example, an environmentally-responsive or bio-responsive hydrogel could be used. Some hydrogels swell in proportion to the amount of water in their environment, and such materials could be used, for example, to monitor the amount of ocular fluid present in a subject's eye at any given time. Hydrogels are also known which respond to changes in pH, temperature, ionic strength and the concentration of specific drugs. Advantageously for biomedical applications, hydrogels are superabsorbent and possess a degree of flexibility very similar to natural tissue.

Environmentally-responsive hydrogels generate a physical response as a result of a change in an environmental factor, e.g., pH, temperature, or the concentration of a metabolite. Hydrogel responses include swelling or collapsing, degradation or erosion, mechanical deformation, optical density variations, and electrokinetic variations. These responses are usually reversible. The particular response exhibited by a given environmentally-responsive hydrogel (i.e. which environmental factor(s) it responds to, and how sharp the response is) can be tailored, e.g. by selecting or engineering a particular polymer or combination of polymers to form the polymer chain network of the hydrogel. For instance, incorporating a polymer having a photo-responsive group can cause a hydrogel to swell/deswell in response to changes in illumination.

Similarly, bio-responsive hydrogels are designed to exhibit a physical response when subjected to a particular biological agent. When the targeted biological agent comes into contact with the hydrogel it is “sensed” by a biorecognition species within the hydrogel, the species being specific to that agent. The biorecognition species can be, for example, a biomacromolecule such as an enzyme, antibody or nucleic acid; any native or synthetic biomimetic variants of the foregoing; or a small molecule such as a metabolite or peptide. When the target biological agent is present in the environment surrounding the bio-responsive hydrogel it diffuses into the hydrogel and causes a perturbation of the thermodynamic equilibrium of the hydrogel system.

In a particular example, a bio-responsive hydrogel includes an immobilized enzyme which catalyzes the conversion of the target agent to a product. The enzymatic reaction is forced away from equilibrium by a change in the chemical potential of the agent, and this manifests as a change in the chemical potential of the product. The change in chemical potential of the product in turn elicits a collapse or swelling of the hydrogel. Examples of biological agents which can trigger engineered responses in a hydrogel include biomolecules (e.g. glucose), large macromolecules (e.g. chymotrypsin), and even whole cells (e.g. vascular endothelial cells). The response can be binary, e.g. presence or absence of the biological agent at a particular threshold limit, or it can scale with the chemical potential or activity of the biological agent. Advantageously for embodiments of the present invention, bio-responsive hydrogels can be designed which produce a measurable response when a specific analyte of biological origin is present.

Another group of materials which are able to exhibit a specific volume response in dependence on environmental factors, and which can therefore be suitable for use as the indicator material, are molecularly imprinted polymers. A molecularly imprinted polymer includes empty sites in the polymer matrix which have an affinity to a specific target molecule. Significant freedom-of-design is possible when creating a molecularly imprinted polymer, meaning that a very wide variety of chemical substances can be targeted. Filling of the empty sites by molecules of the target analyte causes the overall structure of the polymer to change, e.g. through an increase in crosslinking. This structural change generates a volume response.

The contact lens 1 further comprises an output means 12 disposed on the lens part 10. The output means 12 is configured to provide an output which is variable in dependence on the volume of the indicator material. In some embodiments, including the example shown in FIG. 1, the output means comprises an RF antenna in the form of a wire embedded in or fixedly mounted on the indicator material. In such embodiments a volume change of the indicator material causes an alteration of the strain experienced by the antenna wire (i.e. the strain increases as the indicator material expands/swells, and decreases as the indicator material shrinks/deswells). In some alternative embodiments the RF antenna comprises a slotted patch antenna embedded in or fixedly mounted on the indicator material. In such embodiments both the strain in the antenna material and the size of the slot are altered by a volume change of the indicator material.

FIGS. 2a and 2b show a partial cross section of a contact lens according to a first specific embodiment. A first section 22 of the lens part 20 of the contact lens comprises an indicator material and a second section 24 of the lens part 20 comprises a conventional contact lens material. An antenna wire 26 is disposed on the lens part 20 such that it passes over the first section 22. FIG. 2a shows the situation where the indicator material is not experiencing any volume increase compared to a baseline state. FIG. 2b shows the situation where the indicator material has swelled by a measurable amount compared to the baseline state. It can be seen from FIG. 2b that the antenna wire 26 is caused to stretch by the swelling of the indicator material.

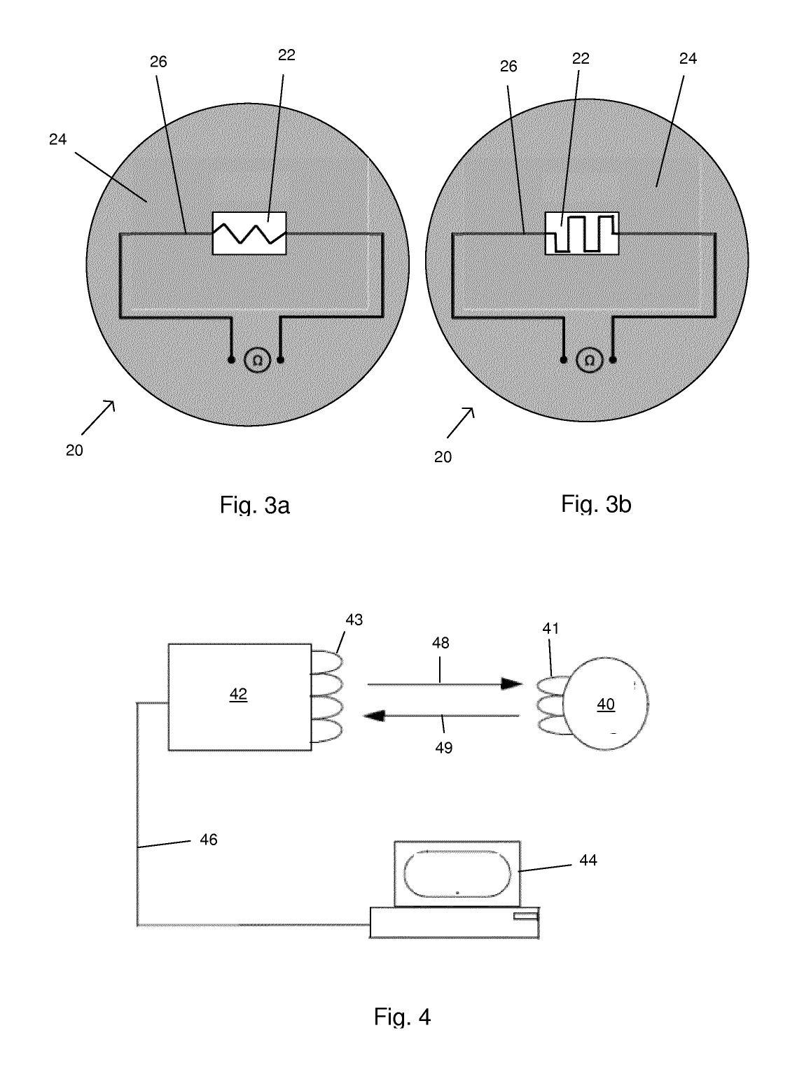

Due to the piezoresistive effect, the resistance of a conductor (such as an antenna wire) varies in dependence on the strain experienced by that conductor. The strain depends on the degree of stretching being experienced by the conductor. Therefore, in embodiments in which the output means comprises an RF antenna, the resistance of the antenna wire varies in dependence on the volume of the indicator material. Changing the resistance of an antenna wire causes changes in the antenna transfer functions (e.g. the resonance frequency of the antenna, the quality factor (QF), etc.). This effect can be amplified by utilising an antenna configuration which experiences a relatively high amount of stretching in response to a given volume increase of the indicator material. FIGS. 3a and 3b show the contact lens of FIG. 2 with antenna wire configurations which amplify the piezoresistive effect experienced by the antenna in response to swelling of the indicator material. For example, arranging the part of the antenna wire which passes over the indicator material in a zigzag (FIG. 3a) or meander (FIG. 3b) pattern enhances the total strain over the whole antenna structure, and therefore increases the resulting change in the antenna transfer functions. This advantageously improves the sensitivity of the contact lens to small volume increases. A similar enhancing effect can also be achieved with a spiral-shaped antenna wire.

In some embodiments the configuration of the antenna is also altered by a volume change of the indicator material. For example, in embodiments in which the antenna wire is arranged in a zig-zag pattern across the indicator material, the distance between adjacent vertices of the zig-zag will increase as the indicator material swells. Such configuration changes will alter the transfer function of the antenna.

Changes to the antenna transfer functions can be detected by a transceiver coupled to the antenna, without requiring contact between the transceiver and the antenna. Advantageously, this means that the sensor output of the contact lens of FIG. 1 can be read remotely, causing little or no discomfort or inconvenience for the user. Furthermore, using an RF antenna as a means for detecting a volume change of the indicator material means that it is not necessary to provide separate means for sensing a volume change and for outputting the sensed result, and nor is it necessary to provide a power source on the contact lens. This advantageously simplifies the device architecture, improving both its cost effectiveness and its unobtrusiveness. For instance, because fewer components need to be disposed on the lens part, it is easier to arrange these components such that they do not interfere with the vision of the user. Also, it is expected that a given contact lens will need to be replaced periodically during an extended monitoring period, e.g. for hygiene reasons, making it particularly desirable for the contact lens to be simple and inexpensive to manufacture, and to dispose of.

Using a measured change in an antenna transfer function, such as QF, it is possible to calculate (using known techniques) the resistance change which caused the observed change in the antenna transfer function. The resistance change will be related to the underlying volume change of the indicator material by a correlation function, the exact form of which will depend on specific factors such as the form of the antenna wire, the form of the indicator material, and the relative arrangement of the antenna wire and the indicator material. In some embodiments a calibration graph or look-up table relating antenna wire resistance to indicator material volume is created in respect of each particular design of the contact lens 1, to enable the volume change of the indicator material to be determined from a calculated resistance change. In some embodiments a processing unit (e.g. comprised in the reader or in communication with the reader) is arranged to determine a correlation function relating resistance change to volume change, and to apply this to the calculated resistance values.

Similarly, the volume of the indicator material will be related to the underlying change of the ocular fluid property by a correlation function, the exact form of which will depend on specific factors such as the nature of the indicator material, the arrangement of the indicator material, and the nature of the property. In some embodiments a calibration graph or look-up table relating indicator material volume to ocular fluid property value is created in respect of each particular design of the contact lens 1, for each property of ocular fluid that can be analyzed using that particular design, to enable the volume change of the indicator material to be determined from a calculated resistance change. In some embodiments a processing unit (e.g. comprised in the reader or in communication with the reader) is arranged to determine a correlation function relating indicator material volume ocular fluid property value, and to apply this to the calculated volume values.

In some alternative embodiments, the output means comprises a strain gauge and a separate RF antenna. It will be appreciated that in some embodiments the output means can be based on the detection of a property other than strain. For instance, in some embodiments conductive plates are disposed on the indicator material such that the distance between the plates is altered by a change in volume of the indicator material. The dielectric constant of the indicator material between the plates will also be altered by a change in volume of the indicator material. The plates thus form a variable capacitor, the capacitance of which depends on the volume of the indicator material. In some embodiments the output means comprises particles of a conductive material (e.g. graphite, gold spheres, etc.) suspended in the indicator material, which in such embodiments is selected to have low or no conductivity. In such embodiments changes in the volume of the indicator material alter the distances between the conductive particles, which in turn alters the conductance indicator material. Various ways of detecting changes in electrical properties such as conductance and/or capacitance suitable for implementing in a contact lens will be known to the skilled person.

In some embodiments specific values for the volume or volume change of the indicator material are not determined. For example, in some such embodiments the RF antenna is arranged to generate a binary on/off response, i.e. such that it responds to an RF signal received from the reader when the volume of the indicator material is less than a threshold volume, and does not respond to an RF signal received from the reader when the volume of the indicator material is greater than or equal to the threshold volume. An antenna exhibiting this type of behaviour can be constructed, for example, by using an antenna wire which has weak/loose connection points (e.g. a conducting wire consisting of conducting particles) which opens up as a consequence of swelling of the indicator material.

It should be appreciated that detected change in a property resulting from volume change of the indicator material can be communicated in various ways other than via an RF antenna. For example, in some embodiments the output means 12 is arranged to generate an indication corresponding to a change in volume of the indicator material, e.g. which can be read by direct inspection of the contact lens. Advantageously, in such embodiments there is no need for a separate reader device to be provided.

FIG. 4 shows a system for detecting changes in a property of ocular fluid, according to a second embodiment of the invention. The system comprises a contact lens 40, and a reader 42. The reader is in communication with a host computer 44 via a communications link 46, which is preferably wireless (but which may, in some embodiments, be wired). Alternative embodiments are envisaged in which all necessary processing capability is provided in the reader, and so a host computer is not required.

The contact lens 40 comprises a lens part, which at least partly comprises an indicator material arranged to change volume in response to a change in a property of ocular fluid. The contact lens 40 also comprises an output means comprising an RF antenna 41 embedded in the lens part such that the strain experienced by the RF antenna depends on the volume of the indicator material. (In FIG. 4 the antenna 41 is shown extending outwardly from the lens part for clarity, but it will be appreciated that this will not be the case in most embodiments). The RF antenna 41 is part of a passive antenna circuit. In some embodiments the RF antenna 41 is tuned to a predefined frequency for a given strain state (i.e. a given volume of the indicator material). In some embodiments the RF antenna 41 and associated circuitry is formed from a transparent conductive material, e.g. indium tin oxide (ITO), so as not to impair the sight of the user. In some embodiments the RF antenna 41 and associated circuitry is arranged around the perimeter of the contact lens 40 so as not to impair the sight of the user. In some embodiments the contact lens has some or all of the features of the contact lens 1 of the first embodiment.

The reader 42 comprises an RF transceiver for transmitting RF energy to and receiving RF energy from the contact lens 40, and a communication interface for communicating with the host computer 44. The RF transceiver comprises an RF signal generator, an antenna 43 and a tuning circuit. In preferred embodiments the transceiver is arranged to transmit RF energy in a frequency including frequencies up to a few tens of MHz. Preferably the transceiver is arranged to transmit RF energy in a range away from commonly used communication bands, and also below the energy absorption range of tissue. Preferably the transceiver is able to be tuned to receive a wide range of RF frequencies (e.g. because the resonance frequency of the contact lens antenna 41 may change in accordance with changes in the ocular fluid property, and because the it is desirable to be able to use the reader 42 to read multiple contact lenses which may vary due to manufacturing tolerances). It is also advantageous for the transceiver to be able to be tuned to receive a wide range of RF frequencies, because it enables a subject to be provided with pair of contact lenses where the left-hand lens is configured to operate at a different frequency to the right-hand lens, without needing an additional reader to also be provided. In some embodiments the transceiver is able to be tuned to receive RF frequencies in the range 100 kHz to 5 GHz.

The communication interface is arranged to convert the received RF energy signal into a data signal and transmit the data signal to the host computer 44. In some embodiments (e.g. embodiments in which the reader is not in communication with a host computer) the reader 42 further comprises a processing unit arranged to determine a value of the property of the ocular fluid based on RF energy received from the contact lens 40. In such embodiments the reader need not comprise a communications interface. In some embodiments the reader is a read-only device. It will be appreciated that the RF signal itself (i.e. sent from the contact lens antenna 41 to the reader 42) will generally not be used to communicate data—instead it is only used to extract an antenna function of the contact lens antenna 41. Indeed, in most embodiments the contact lens is not configured to hold any data or perform any signal processing by which data may be generated.

A read range may be defined as the maximum distance between the reader 42 and the contact lens 40 at which the reader is able to receive useful (i.e. having a sufficiently low signal to noise ratio) RF energy from the tag. The distance at which the reader is able to receive useful RF energy from the tag will vary together with the antenna transfer functions, in particular the QF. A minimum read range, corresponding to a lowest possible QF of the contact lens antenna 41, can therefore be defined. In some embodiments the minimum read range is of the order of a few millimetres. The reader 42 is arranged to transmit a small RF pulse at a dynamic frequency (e.g. a chirp), in order to track the changes in the antenna transfer functions of the contact lens RF antenna 41.

In some embodiments the reader 42 is a hand-held device. In some embodiments the reader is incorporated into a portable electronic device such as a smartphone or tablet computer. In some embodiments the reader 42 is configured to be worn on a body part of the user, e.g. on a wrist or around the neck, etc. In some embodiments the reader is configured to be mounted to a pair of spectacles. In some embodiments the reader 42 is integrated into a pair of spectacles. In such embodiments the spectacles may, but need not, have vision-correcting power.

The host computer 44 comprises a communication interface for sending and receiving communications signals to/from the reader 42 and a processing unit. The processing unit is arranged to determine a value of the property of the ocular fluid based on a communications signal received from the reader 42, the received communications signal being based on RF energy received from the contact lens 40. In some embodiments the processing unit is arranged to send a control signal (via the communications interface) to the reader 42. The control signal may, for example, cause the reader to begin transmitting RF energy, to stop transmitting RF energy, and/or change a parameter of its transmission of RF energy. In some embodiments he processing unit is arranged to determine a value of the property of the ocular fluid by measuring a transfer function of the contact lens RF antenna 41. In some such embodiments the processing unit is arranged to measure a transfer function of the antenna 41 at a first time and at a second, later, time. The measured transfer function can comprise any of: a quality factor (QF), a resonance frequency, harmonics of a resonance frequency, time constants of an RLC circuit of the antenna.

In some embodiments the processing unit is arranged to determine a resistance of the contact lens RF antenna 41 based on the measured transfer function. In some embodiments the processor is arranged to determine a volume of the indicator material in the contact lens 40 based on a determined resistance of the contact lens RF antenna 41, e.g. by comparing a determined resistance value to a calibration graph or look-up table relating antenna wire resistance to indicator material volume. In some embodiments the processing unit is arranged to determine a value of a ocular fluid property (e.g. concentration of the target analyte) based on a determined volume of the indicator material, e.g. by comparing a determined indicator material volume to a calibration graph or look-up table relating indicator material volume to ocular fluid property value.

The strain experienced by the contact lens antenna 41 (and therefore the antenna transfer function) can be altered by the subject blinking. Therefore, in some embodiments the processing unit is arranged to correct the signal received from the contact lens 40 to account for changes which are caused by blinking rather than by a change in an ocular fluid property. In some embodiments the processing unit is arranged to correct the signal by filtering out a repetitive signal that corresponds to an expected range of blinking frequency and amplitude. In some alternative embodiments the processing unit is arranged to control the reader to transmit RF energy only when the subject's eye is open, so that it is not necessary to correct the signal to remove the effects of blinking. This can be achieved, for example, by providing the reader 42 with a camera arranged to view the subject's eye, arranging the processing unit to detect whether the subject's eye is open or closed based on data received from the camera, and/or arranging the processing unit to control the transmission of RF energy from the reader in dependence on the detected state of the subject's eye.

In some embodiments the reader 42 comprises a processing unit arranged to perform some or all of the functions described above in relation to the host computer processing unit.

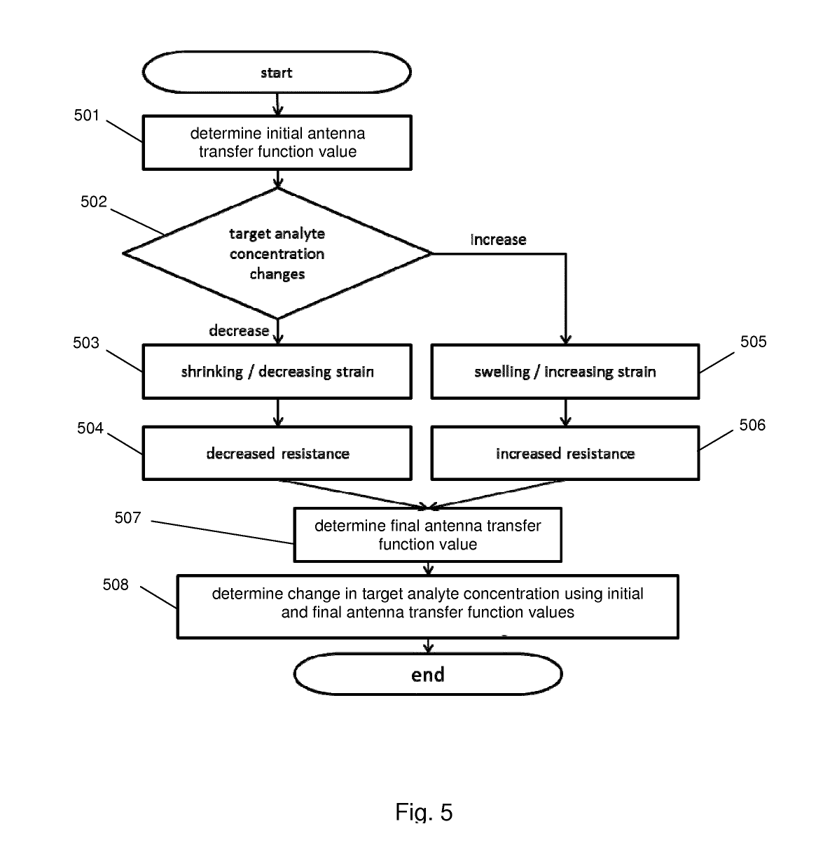

The operation of the system of FIG. 4 will be further described with reference to FIG. 5, which illustrates a method for determining the concentration in ocular fluid of a target analyte. The target analyte could be, for example, glucose, an amino acid (glutamate, etc.), an organic acid (lactate, pyruvate, etc.), a fatty acid, a polyol (glycerol, etc.), a hormone (e.g. melatonin, cortisol, etc.), a protein, a metabolite, an enzyme, a nucleic acid, a lipid, an electrolyte, a chemical induced by medication intake, and/or an environmental pollutant/chemical.

In some embodiments, prior to performing the first block 501 of the method, a resonance frequency of the contact lens antenna 41 is determined. This is advantageous in cases where the resonance frequency of the contact lens antenna 41 is not be known exactly, e.g. because of manufacturing tolerances, thermal changes in the eye (which can cause slight changes to the resonance frequency), or because the reader 42 is arranged to be suitable for use with contact lens antennas having various different resonance frequencies. The resonance frequency of the contact lens antenna 41 can be determined by sweeping the RF transmission from the reader 42 over a range of frequencies, and finding the frequency for which the response signal has the highest amplitude. In some embodiments the reader 42 is locked to the resonance frequency of the contact lens antenna 41. In some embodiments the resonance frequency is determined at a later stage of the method shown in FIG. 5.

In a first block 501 of the method the reader 42, using the transceiver module, measures an antenna transfer function at a first time, to determine an initial value for that antenna transfer function. During the performance of block 501 the reader 42 is positioned such that the distance between the reader 42 and the contact lens 40 is less than a maximum read range of the reader 42. The measuring comprises the reader 42 transmitting (using the antenna 43) RF energy in the direction of the contact lens 40. In some embodiments the frequency of the transmitted RF energy is in the range 13-14 MHz. In some embodiments the transmitted RF energy comprises a pulse having a duration and a variable frequency over the duration. In some embodiments the transmitted RF energy is varied between at least two different frequencies. In some embodiments the transmitted RF energy is varied between three different frequencies. In some embodiments the transmitted RF energy is varied over a continuous range of frequencies. The measuring further comprises the antenna 41 of the contact lens 40 receiving the RF energy transmitted by the reader 42.

The RF energy received by the contact lens antenna 41 induces an RF voltage in the contact lens antenna 41, which causes the contact lens antenna 41 to emit RF energy. The RF energy emitted by the contact lens antenna 41 is then received by the reader antenna 43. The RF voltage in the contact lens antenna 41 is linked to the RF voltage in the reader antenna 43, such that the two antennas are coupled in a manner similar to weakly coupled transformer coils. A characteristic relating to the RF signal received by the reader antenna 43 is detected and recorded by the reader 42. In some embodiments the characteristic comprises the amplitude of the received RF signal. In some embodiments the characteristic comprises the voltage in the reader antenna 43. In some embodiments the characteristic is continuously detected and recorded for at least the duration over which the RF energy was transmitted by the reader. In some embodiments the reader generates a time-series of values of the characteristic.

The measuring further comprises calculating a value of the transfer function based on the characteristic relating to the received RF signal. In some embodiments the calculating is performed by a processing unit of the reader 42. In some embodiments the reader transmits amplitude data to a separate device, e.g., the host computer 44, and the calculating is performed by a processing unit of the separate device.

In a particular example in which the transfer function comprises a QF and the characteristic comprises the amplitude of the received RF signal, the calculating is performed as follows. The QF describes the width of the frequency spectrum of an antenna at 3 dB below the peak. To calculate a QF it is therefore necessary to measure the spectrum of the received RF energy at at least two frequencies. A suitable calculation process comprises:
determining the maximum amplitude of the received signal and a corresponding frequency, f₀, of the transmitted signal;
determining a first frequency, f₁, of the transmitted signal corresponding to an amplitude 3 dB less than the maximum amplitude;
determining a second frequency, f₂, of the transmitted signal corresponding to an amplitude 3 dB less than the maximum amplitude; and
calculating a QF value using:

$$QF = \frac{{f}_{0}}{{f}_{2} - {f}_{1}} . \qquad ( Equation 1 )$$

However; it is often the case that the shape of the antenna band-pass characteristic (in particular the fact that it is symmetric) is known. In such cases f₂−f₀=f₀−f₁, meaning that it is only necessary to determine the peak frequency f₀, and one of the −3 dB frequencies (either f1 or f2).

In examples in which the characteristic comprises the voltage in the reader antenna, the calculating process is slightly different. In such examples the maximum voltage is determined and this value is multiplied by 0.707 in order to obtain the equivalent −3 dB value. The frequencies corresponding to the maximum voltage and the −3 dB equivalent voltage are then determined and input into equation 1.

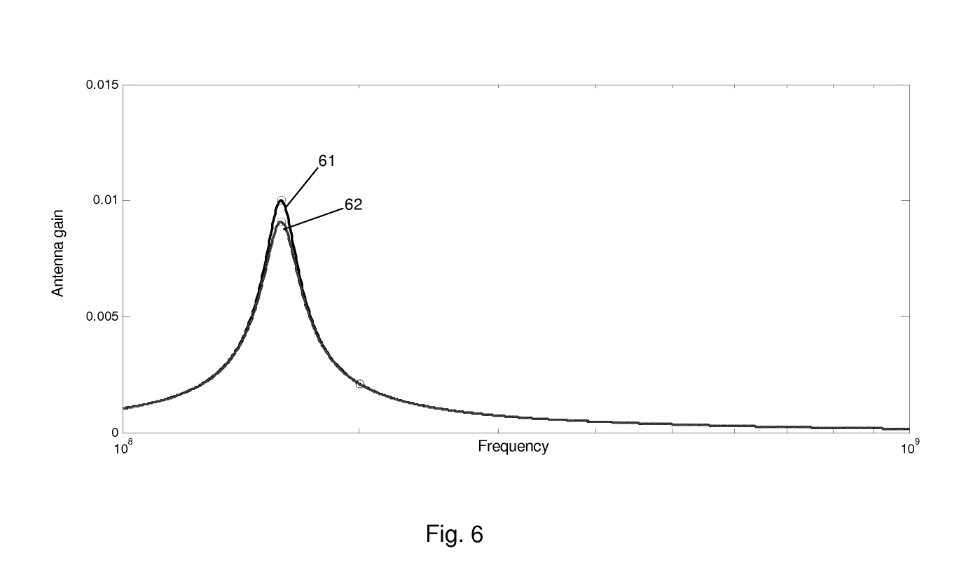

When an initial value for the antenna transfer function has been determined, the method moves to block 502 in which the concentration of the target analyte in the ocular fluid changes. It will be appreciated that block 502 occurs in the eye, and is not a step in the operation of the system. The change can comprise either an increase or a decrease in concentration. The lens part (i.e. the round, transparent part which is arranged to mount to an eye) of the contact lens 40 comprises an indicator material which is arranged to shrink in response to a decrease in concentration of the target analyte, and to swell in response to an increase in concentration of the target analyte. Thus, if the change in block 502 is a decrease in concentration, in block 503 the indicator material in the contact lens 40 shrinks and this reduces the strain experienced by the antenna wire of the contact lens antenna 41. The reduced strain in turn causes the resistance of the antenna wire to decrease, block 504. If, on the other hand, the change in block 502 is an increase in concentration, in block 505 the indicator material in the contact lens 40 swells and this increases the strain experienced by the antenna wire of the antenna 41. The increased strain in turn causes the resistance of the antenna wire to increase, block 506. The change in the resistance of the antenna wire causes the transfer function of the contact lens antenna 41 to change. An example antenna gain response (antenna gain correlates with QF) to a 10% resistance change in the antenna is shown in FIG. 6. It can be seen from this figure that an antenna with baseline resistance (line 61) has a higher gain than an antenna with a 10% higher resistance (line 62).

Thus, in block 507 the reader 42 measures the antenna transfer function at a second time, to determine a final value for that antenna transfer function (the term “final” is used merely to distinguish this value from the initial value, and is not intended imply that no further values of the antenna transfer function are determined). The determination of the final antenna transfer function value is performed in the same manner as the determination of the initial antenna transfer function value. In some embodiments the second time is immediately after the first time, i.e. such that the reader is continuously determining an updated antenna transfer function value. In some embodiments there is a period between the first time and the second time, and the duration of the period is of the order of a few milliseconds. However, the duration of the period can range from a few milliseconds to several hours, depending on the requirements of the particular application for which the contact lens is being used.

In block 508 a processing unit, e.g. in the reader 42 or in the host computer 44 in communication with the reader 42, determines a change in the concentration of the target analyte between the first time and the second time using the initial antenna transfer function value and the final antenna transfer function value. In some embodiments the determining comprises calculating a resistance change which caused the observed change in the antenna transfer function. In some embodiments the determining comprises calculating a change in the strain experienced by the contact lens antenna 41 based on the initial and final antenna transfer function values. In some embodiments the determining comprises determining a volume change of the indicator material, e.g. using a calibration graph or look-up table relating antenna wire resistance to indicator material volume, or relating antenna wire strain to indicator material volume. In some embodiments the determining comprises determining a concentration change of the target analyte based on the determined volume change, e.g. using a calibration graph or look-up table relating indicator material volume to target analyte concentration. In some embodiments the determining comprises determining a correlation function relating resistance change to volume change, and applying this to calculated resistance values. In some embodiments the determining comprises determining a correlation function relating strain change to volume change, and applying this to calculated strain values. In some embodiments the determining comprises determining a correlation function relating volume change to concentration of the target analyte, and applying this to calculated volume values. It will be appreciated that the initial antenna transfer function and the final antenna transfer function used in step 508 do not have to be consecutive measurements of the antenna transfer function value. For example, if antenna transfer functions are measured frequently (e.g. of the order of milliseconds or a few seconds), then a change in the concentration of the target analyte over a period of several hours or days can be determined using the most recent antenna transfer function value and an antenna transfer function value measured several hours or days earlier.

In some embodiments the method comprises an optional further step of outputting the determined concentration change. In such embodiments the outputting may comprise one or more of:

displaying a concentration value and/or trend on a display of the reader;—

displaying a subject status on a display of the reader;

displaying a concentration value and/or trend on a display of the host computer;

displaying a subject status on a display of the host computer;

sending a message containing concentration information and/or subject status information to a device of a caregiver;

sending a message containing concentration information and/or subject status information to a device of the subject;

storing a concentration value in a memory;

sending a concentration value to a remote server;

sending a signal based on the determined concentration change to a medication administration device.

It will be appreciated that any suitable display form can be used to display information such as a concentration value, trend, subject status, etc. Suitable display forms include, for example, numerical values, graphs, color-coded pictograms, text, etc. It is expected that the use of contact lenses and systems according to embodiments of the invention could be beneficial in the following areas:
Monitoring and management of therapeutic treatment for various conditions, including hypertension, heart failure and respiratory disorders;
assessment of patient compliance and adherence to treatment regimes through the detection of therapeutic drugs such as Phenobarbital, Carbamazepine, and Methotrexate;
monitoring and management of ocular side effects of therapeutic drugs, such as the condition “dry eye”;
monitoring the health status and general well-being of a subject (user) with a known disease/condition (e.g. by monitoring drug and/or disease markers)—ocular fluid analysis can replace traditional biofluidic analyses;
screening for an unknown disease/condition, e.g. by the detection in ocular fluid of disease markers.
monitoring the effect of environmental conditions, such as ambient air pollution, toxic chemical exposure, etc.

### Fertility Testing

In addition to use in monitoring and managing therapeutic treatment for various condition, the contact lens according to the invention can be adapted or used as or as part of a fertility test to help women estimate the relatively fertile and relatively infertile days of their menstrual cycle.

In particular, the contact lens according to the invention can be used as part of a method for ovulation detection which continuously or semi-continuously measures properties of the ocular fluid for signs of ovulation, optionally in conjunction with other apparatus for measuring the user for other signs of ovulation. The signs that can be measured using the contact lens include hormone and/or salt concentration, blinking frequency and body temperature (although optionally one or more of these can be measured using a separate apparatus). Applying a contact lens which can measure the hormonal level and/or other characteristics/signs is easy to do and easy to remember, and thus provides a more convenient way to estimate the timing of ovulation than conventional fertility tests that, for example, require specific action by the user to perform a test.

By using a contact lens, it is possible to make measurements at the most optimal point of time during the day/night (and several times a day), and log the hormone and/or salt levels and temperature in order to find a trend consistent with the occurrence of an ovulation. The measurements can then be analysed to provide the user with information on their fertility window.

A fertility testing system can comprise one or two contact lens(es), that can each or individually perform or enable one or more of the following: tear sampling; hormone concentration analysis; salt concentration analysis; temperature measurement; and blinking frequency measurement. The analysis of these measurements can be performed in the contact lens itself, or by a separate device, and the feedback about the user's fertility window can be provided via that external device (e.g. on a display), or visual feedback can be provided via the contact lens itself. Where the contact lens(es) only measure some of the above, it is possible for the system to comprise other apparatus to measure one or more of the other parameters.

In a first particular embodiment, a contact lens is used for fertility testing in which hormone level measurements and temperature measurements are combined. A contact lens 1 according to the present invention is used to determine or measure trends in the female reproductive hormones, repeatedly over night and day. Furthermore, in this embodiment the contact lens 1 is also configured to measure the temperature of the user repeatedly throughout the day and night.

The data on the hormone levels combined with the temperature data can be collected from the contact lens 1 according to the embodiments described above, and the data used in an algorithm that determines the most likely time for ovulation, and that detects actual ovulation taking place. This information can be used by the user to schedule sexual intercourse and/or the use of birth control measures to increase or decrease the chance of conception, based on the user's preference.

As noted above, hormone levels in the tears can be detected through the use of an indicator material whose volume changes in response to the hormone level.

Temperature is optimally measured first thing in the morning (or after the longest sleep period of the day), even before getting out of bed. The best (i.e. most useful) results are obtained by measuring the temperature every day at the same time, before eating or drinking ideally, the core body temperature is measured. The temperature in the eye is influenced by the core body temperature. However, when the eyes are open the eye temperature is also affected by ambient temperature, humidity, air flow, etc. The temperature of the eye/tears with the eyelids closed is more likely to follow the trend of the core body temperature. The basal temperature is the lowest temperature of the body during a 24-hour period, and it is usually reached during sleep. The basal temperature is the temperature that is most useful for predicting ovulation.

Therefore the temperature can be measured by several methods: during sleep (in order to obtain the best estimate of the basal temperature), and/or when the user is asked to close her eyes during the day.

In some embodiments the temperature is measured in the contact lens 1 with a thermocouple or other temperature-sensitive electronic component, or with a material that swells/shrinks upon a temperature change (and for example which is measured with a strain sensing antenna as described above). Alternatively, the resistance of the wires in the contact lens 1 (e.g. antenna wire 26 or an additional wire) can also be used as a temperature indicator.

If necessary, the in-eye temperature measurement can be correlated to the core body temperature by an extra calibration measurement using standard temperature measurement devices, such as rectal or in-ear thermometers.

In some embodiments, the precision of the ovulation detection can be improved by measuring the levels of multiple types of hormones using the contact lens 1 (or by measuring a first hormone with a first contact lens 1 in the left eye and a second hormone with a second contact lens 1 in the right eye). The most important hormones to measure are follicle stimulating hormone (FSH), luteinizing hormone (LH), estrogen and progesterone.

Progesterone and estrogen (17β estradiol) are known to be present in tears, and it is known that the progesterone concentration in tears varies due to ovulation. A surge or lack of surge of the concentration of progesterone in blood/serum is reflected in the tear film concentrations of progesterone.

Analyzing the concentrations of two or more hormones in tears will make the prediction and actual detection of ovulation more precise. The reliability of the ovulation test can be increased by detecting (at least) two hormones since it enables:

a. measurements of two hormones to show contradictory behaviour (e.g. one increases, while the other decreases). For example an increase in progesterone and a decrease in the other hormones happens at the ovulation;

b. the use of the ratio between two hormone levels as an indicator of the fertility, for example the ratio between progesterone and estrogen;

c. the use of (at least) one hormone level for compensating background level changes, for example the progesterone concentration is always low in the follicular phase;

d. the use of the secondary (or tertiary) analyte as a confirmation of the trend observed for the first target analyte. For example both FSH and LH levels should spike just prior to ovulation.

The use of a contact lens 1 to detect hormone levels in the tears is already mentioned above. In addition, detection of progesterone can also or alternatively be done using an electrochemical biosensor or Electrochemical Impedance Spectroscopy (EIS); and the detection of estrogen can also or alternatively be done using an electrochemical sensor or a nanoporous polymeric film.

In a second particular embodiment of the fertility testing system, the precision of the fertility testing can be improved by measuring the tear salt concentration in addition to the hormone levels and temperature in the first particular embodiment.

It is known that the hormonal changes around ovulation also alter the salt concentration in body fluids such as saliva. The addition of data on the salt concentration in tears may further improve the precision of prediction and actual detection of ovulation.

Salt concentration in tears can be detected by several methods, such as conductivity of the tears, surface tension (hence tear film stability), evaporation rate, drying pattern and viscosity. Also the indicator material in the contact lens 1 can swell/shrink as a function of changes in salt concentration.

In a third particular embodiment, the precision of the fertility testing can be improved by measuring the blinking rate of the user. This embodiment can be combined with either of the first and second particular embodiments described above. The blinking rate can be measured using the contact lens 1, or via another sensor.

In particular, blinking can be detected using the features provided for strain and/or temperature sensing described above, since a sudden resistance change in the sensing wires may occur due to the strain during blinking, and this resistance change can be detected. Alternatively blinking can be detected using a light sensor that is embedded in the contact lens 1.

It has been found that the blinking frequency decreases substantially (e.g. from 13 to 2 times per minute) upon a drop in estrogen level. In particular, the blinking frequency for women who are not taking birth control pills decreases in week 2 and week 4 (with menstruation being considered week 0). In both week 2 and 4, the estrogen level drops in the menstrual cycle. The data about the blinking frequency (e.g. absolute blinking frequency or current blinking frequency compared to the blinking frequency in previous days and/or weeks) can further improve the reliability of the fertility test described in the previous embodiments.

Combining the blinking frequency information and the measured hormone levels provides information about which phase of the menstrual cycle the user is in (since a blinking frequency decrease can indicate both the ovulation and the start of the menstruation).

Additionally, the blinking rate may be used to improve the temperature measurement by a contact lens (embodiment 1). Using the blinking rate detection part of the lens we can easily measure if a person is asleep or has their eyes closed for a prolonged period of time, allowing for a reliable measurement in the eye. Because the blinking reflex is impossible to supress when the eyes are open we can simply look at the lack of blinking over a certain time frame and therefore we can assume that the eyes are closed during that period.

In a fourth particular embodiment, the fertility test can be performed using a contact lens 1 in combination with other devices. For example, the contact lens 1 can measure the hormone level and/or the salt concentration, but the temperature measurement can be performed with a separate device (e.g. a rectal or in-ear thermometer).

There is therefore provided a contact lens (and optionally, an associated reader) which can non-invasively detect changes in one or more properties of ocular fluid, and which is suitable for continuously analyzing ocular fluid over a period of time.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A contact lens for detecting changes in a property of ocular fluid, the contact lens comprising:
a lens part comprising an indicator material, wherein the volume of the indicator material is variable in dependence on a property of ocular fluid; and
a RF antenna disposed on the indicator material such that a change in the volume of the indicator material causes a change in the strain experienced by a conductive part of the RF antenna and/or a change in the configuration of the RF antenna, such that a signal transmitted by the RF antenna is variable in dependence on the volume of the indicator material, wherein a part of the RF antenna that passes over the indicator material is arranged in a zigzag pattern, a meander pattern or a spiral-shaped pattern.

2. A contact lens according to claim 1, wherein:
the property comprises one or more of: the presence of a target analyte, the concentration of a target analyte, pH, volume, osmolarity, a ratio of compounds in the ocular fluid; evaporation rate; viscosity; rheology; tear film stability; temperature; density.

3. A contact lens according to claim 2, wherein:
the property comprises the concentration of a target analyte;
the indicator material is arranged to absorb the target analyte; and
the volume of the indicator material is variable in dependence on the amount of the target analyte contained in the indicator material.

4. A contact lens according to claim 3, wherein the target analyte comprises one of: glucose; an amino acid; an organic acid; a fatty acid, a polyol; a hormone; a protein, a metabolite, an enzyme, a nucleic acid, a lipid, an electrolyte, a chemical induced by medication intake; an environmental pollutant.

5. A contact lens according to claim 1, wherein the indicator material comprises one or more of:
a bio-responsive material, wherein the volume of the bio-responsive material is variable in dependence on the presence and/or concentration of a target biological agent;
an environmentally-responsive material, wherein the volume of the environmentally-responsive material is variable in dependence on an environmental factor.

6. (canceled)

7. A contact lens according to claim 1, wherein the RF antenna is configured such that a transfer function of the RF antenna is variable in dependence on the strain experienced by the conductive part of the RF antenna.

8. A system for detecting changes in a property of ocular fluid, the system comprising:
a contact lens according to claim 1; and
a reader arranged to:
transmit RF energy to the contact lens; and
in response to transmitting RF energy to the contact lens, receive RF energy from the RF antenna; and
a processing unit arranged to:
measure a transfer function of the RF antenna from the received RF energy; and
detect a change in the property of the ocular fluid based on the measured transfer function.

9. A system according to claim 8, wherein the reader is arranged to receive the RF energy without being in contact with the contact lens.

10. A system according to claim 8, wherein the processing unit is arranged to determine a value of the property based on the detected change in the property.

11. A system according to claim 10, wherein the reader is arranged to continuously detect the received RF energy and the processing unit is arranged to determine a time-series of values of the property.

12. A system according to claim 10, wherein the processing unit is further arranged to generate at least one output signal based on the determined value, the at least one output signal comprising one or more of:
a signal arranged to cause the determined value to be shown on a display of the reader;
a signal arranged to cause the determined value to be shown on a display of a remote device;
a message to a portable device of a caregiver containing the determined value;
a data transmission to a memory of the reader;
a data transmission to a remote server.

13. A system according to claim 10, wherein the processing unit is comprised in the reader.

14. A system according to claim 10, wherein the system is for determining a measure of the fertility of a user, wherein the property of the ocular fluid is one or more of a hormone level, salt concentration and body temperature, and wherein the processing unit is arranged to determine a measure of the fertility of the user from the detected change in the property of the ocular fluid.

15. A system according to claim 14, wherein the system is for estimating the timing of ovulation in the user.

16. A system according to claim 10, wherein the system is for determining a measure of the fertility of a user, wherein the properties of the ocular fluid are hormone level and temperature, and wherein the processing unit is further arranged to:
receive an indication of the blinking rate of the user;
determine from the blinking rate if the user is asleep or has their eye closed for a prolonged period of time; and
determine the temperature of the ocular fluid when the user is determined to be asleep or has their eye closed for a prolonged period of time.
